(19) United States
(12) Patent Application Publication
(10) Pub. No.: US 2025/0248657 A1
(43) Pub. Date: Aug. 7, 2025

(54) MOTION ARTIFACT MITIGATION IN SEISMOCARDIOGRAPHY AND GYROCARDIOGRAPHY

(71) Applicant: THE ALFRED E. MANN FOUNDATION FOR SCIENTIFIC RESEARCH, Valencia, CA (US)

(72) Inventor: Jason GOLDBERG, Oakland, CA (US)

(21) Appl. No.: 19/038,692

(22) Filed: Jan. 27, 2025

Related U.S. Application Data
(60) Provisional application No. 63/548,738, filed on Feb. 1, 2024.

(51) Int. Cl. A61B 5/00; A61B 5/11; G16H 40/63

(52) U.S. Cl. CPC A61B 5/7214; A61B 5/1102; A61B 5/725; G16H 40/63

(57) ABSTRACT

A system and method for motion artifact mitigation in seismocardiography and gyrocardiography. In some embodiments, a method includes: obtaining a first measured motion signal from a subject, the first measured motion signal being measured along a first direction; obtaining a second measured motion signal from the subject, the second measured motion signal being measured along a second direction different from the first direction; and generating a first calculated cardiac motion signal, the generating including combining the first measured motion signal and the second measured motion signal.

### CROSS-REFERENCE TO RELATED APPLICATION(S)

The present application claims priority to and the benefit of U.S. Provisional Application No. 63/548,738, filed Feb. 1, 2024, entitled “MOTION ARTIFACT CANCELLATION IN SEISMOCARDIOGRAPHY AND GYROCARDIOGRAPHY”, the entire content of which is incorporated herein by reference.

### FIELD

One or more aspects of embodiments according to the present disclosure relate to biomarker monitoring, and more particularly to motion artifact mitigation in seismocardiography and gyrocardiography.

### BACKGROUND

Inertial sensors, such as accelerometers and gyroscopes, may be employed to monitor various biomarkers of a subject. For example, inertial sensors may be used to detect heart beats and to calculate the heart rate of the subject.

It is with respect to this general technical environment that aspects of the present disclosure are related.

### SUMMARY

According to an embodiment of the present disclosure, there is provided a method, including: obtaining a first measured motion signal from a subject, the first measured motion signal being measured along a first direction; obtaining a second measured motion signal from the subject, the second measured motion signal being measured along a second direction different from the first direction; and generating a first calculated cardiac motion signal, the generating including combining the first measured motion signal and the second measured motion signal.

In some embodiments, the first direction is substantially along a dorsal-ventral axis of the subject.

In some embodiments, the second direction is substantially perpendicular to the first direction.

In some embodiments, the method further includes obtaining a third measured motion signal from the subject, the third measured motion signal being measured along a third direction different from the first direction and different from the second direction, wherein the generating includes combining: the first measured motion signal, the second measured motion signal, and the third measured motion signal.

In some embodiments, the combining of the first measured motion signal and the second measured motion signal includes processing the second measured motion signal with a filter.

In some embodiments, the method further includes adjusting one or more parameters of the filter.

In some embodiments, the adjusting includes adjusting the one or more parameters using least mean squares filtering based on the first calculated cardiac motion signal.

In some embodiments, the method further includes processing the first calculated cardiac motion signal with a first filter to generate a second calculated cardiac motion signal, the first filter having characteristics related to: a model cardiac motion signal; and a calculated noise signal.

In some embodiments, the first filter includes: a whitening filter; and a matched filter.

In some embodiments, the method further includes processing the first calculated cardiac motion signal with a first filter to generate a calculated subject motion signal, the first filter including a first notch filter for suppressing signal components at or near a first harmonic of a calculated heart rate frequency.

In some embodiments, the first filter includes a second notch filter for suppressing signal components at or near a second harmonic of the calculated heart rate frequency.

In some embodiments, the method further includes: applying group delay compensation; and calculating a difference between a first signal, based on the first calculated cardiac motion signal, and a second signal, based on the calculated subject motion signal.

In some embodiments, the method further includes: processing the first calculated cardiac motion signal with a first filter; and processing the first calculated cardiac motion signal with a second filter, wherein: the first filter is configured to pass a first harmonic of a first hypothetical heart rate; and the second filter is configured to pass a first harmonic of a second hypothetical heart rate.

In some embodiments, the method further includes: calculating a covariance matrix of the first calculated cardiac motion signal; and calculating a plurality of filter coefficients of the first filter based on the covariance matrix.

In some embodiments: the calculating of the filter coefficients includes calculating coefficients that minimize an output power, subject to passing, with unit gain, the first harmonic of the first hypothetical heart rate; and the output power is a matrix product including, as factors, the covariance matrix, and a vector including the coefficients.

In some embodiments, the calculating of the covariance matrix includes calculating the covariance matrix based on: a previously calculated covariance matrix, and samples of the first calculated cardiac motion signal.

According to an embodiment of the present disclosure, there is provided a system, including: an inertial sensor; and a processing circuit, the processing circuit being configured: to obtain a first measured motion signal from the inertial sensor, the first measured motion signal being measured along a first direction; to obtain a second measured motion signal from the inertial sensor, the second measured motion signal being measured along a second direction different from the first direction; and to generate a first calculated cardiac motion signal, the generating including combining the first measured motion signal and the second measured motion signal.

In some embodiments, the system is configured to be secured to a subject, with the first direction substantially along a dorsal-ventral axis of the subject.

In some embodiments, the second direction is substantially perpendicular to the first direction.

In some embodiments, the processing circuit is further configured to obtain a third measured motion signal from the inertial sensor, the third measured motion signal being measured along a third direction different from the first direction and different from the second direction, wherein the generating includes combining: the first measured motion signal, the second measured motion signal, and the third measured motion signal.

In some embodiments, the combining of the first measured motion signal and the second measured motion signal includes processing the second measured motion signal with a filter.

In some embodiments, the processing circuit is further configured to adjust one or more parameters of the filter.

In some embodiments, the adjusting includes adjusting the one or more parameters using least mean squares filtering based on a time-averaged power of the first calculated cardiac motion signal.

In some embodiments, the processing circuit is further configured to process the first calculated cardiac motion signal with a first filter to generate a second calculated cardiac motion signal, the first filter having characteristics related to: a model cardiac motion signal; and a calculated noise signal.

In some embodiments, the first filter includes: a whitening filter; and a matched filter.

In some embodiments, the processing circuit is further configured to process the first calculated cardiac motion signal with a first filter to generate a calculated subject motion signal, the first filter including a first notch filter for suppressing signal components at or near a first harmonic of a calculated heart rate frequency.

In some embodiments, the first filter includes a second notch filter for suppressing signal components at or near a second harmonic of the calculated heart rate frequency.

In some embodiments, the processing circuit is further configured to: apply group delay compensation; and calculate a difference between a first signal, based on the first calculated cardiac motion signal, and a second signal, based on the calculated subject motion signal.

In some embodiments, the processing circuit is further configured to: process the first calculated cardiac motion signal with a first filter; and process the first calculated cardiac motion signal with a second filter, wherein: the first filter is configured to pass a first harmonic of a first hypothetical heart rate; and the second filter is configured to pass a first harmonic of a second hypothetical heart rate.

In some embodiments, the processing circuit is further configured to: calculate a covariance matrix of the first calculated cardiac motion signal; and calculate a plurality of filter coefficients of the first filter based on the covariance matrix.

In some embodiments: the calculating of the filter coefficients includes calculating coefficients that minimize an output power, subject to passing, with unit gain, the first harmonic of the first hypothetical heart rate; and the output power is a matrix product including, as factors, the covariance matrix, and a vector including the coefficients.

In some embodiments, the calculating of the covariance matrix includes calculating the covariance matrix based on: a previously calculated covariance matrix, and samples of the first calculated cardiac motion signal.

### BRIEF DESCRIPTION OF THE DRAWINGS

These and other features and advantages of the present disclosure will be appreciated and understood with reference to the specification, claims, and appended drawings wherein:

FIG. 1 is a block diagram of a system for seismocardiography or gyrocardiography with motion artifact mitigation, according to an embodiment of the present disclosure;

FIG. 2 is a block diagram of a system with motion artifact mitigation based on different sensitivity, to cardiac motion, in different axes, according to an embodiment of the present disclosure;

FIG. 3 is a block diagram of a system with motion artifact mitigation based on adaptive filtering, according to an embodiment of the present disclosure;

FIG. 4 is a block diagram of a system with motion artifact mitigation based on suppression of heart rate harmonics, according to an embodiment of the present disclosure;

FIG. 5 is a block diagram of a system with motion artifact mitigation based on a bank of filters, according to an embodiment of the present disclosure;

FIG. 6A is a first portion of a flow chart, according to an embodiment of the present disclosure; and

FIG. 6B is a second portion of a flow chart, according to an embodiment of the present disclosure.

### DETAILED DESCRIPTION

The detailed description set forth below in connection with the appended drawings is intended as a description of exemplary embodiments of a system and method for motion artifact mitigation in seismocardiography and gyrocardiography provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

Chest-mounted or chest-implanted microelectromechanical systems (MEMS) inertial sensors may output signals which may be used to make inferences about cardiac state. For example, signals from a three-axis accelerometer may be used to calculate a seismocardiogram (SCG), which in turn may be used to track instantaneous heart rate (HR) or respiration rate (RR). Likewise, signals from a three-axis gyroscope may be used to calculate a so-called gyrocardiogram (GCG) which, alone or in conjunction with the seismocardiogram, can also be used to track heart rate.

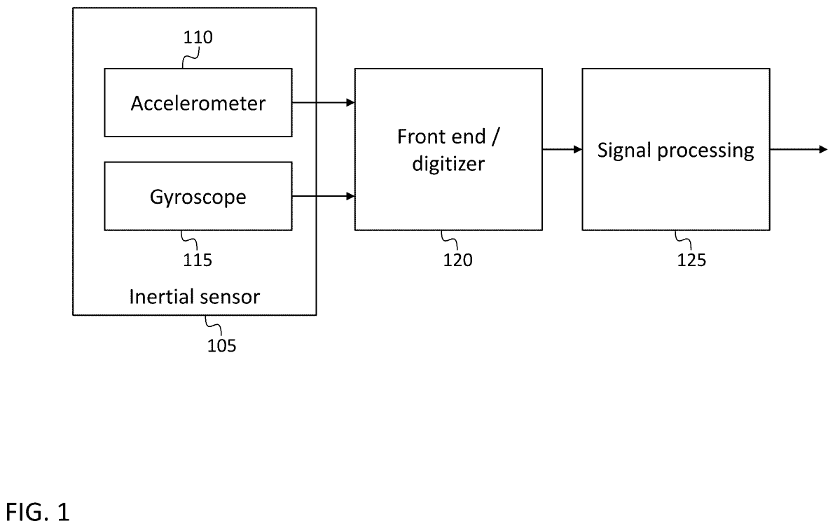

FIG. 1 shows an inertial sensor 105 including an accelerometer 110 (e.g., a three-axis accelerometer) and a gyroscope 115 (e.g., a three-axis gyroscope), connected to a front end and digitizer circuit 120 (which may include one or more analog preamplifiers and one or more analog to digital converters) and a signal processing circuit 125 (which may be a processing circuit (discussed in further detail below)). A system like that shown in FIG. 1, or a portion of such a system (e.g., the inertial sensor 105) may be secured to the chest of a subject, or implanted in the subject, and used to obtain seismocardiograms or gyrocardiograms.

Both the seismocardiogram and the gyrocardiogram may be highly sensitive to subject motion (e.g., motion of the patient, or “subject”), such as standing up, walking, or sitting down. Inertial sensor signals (which may be referred to as “measured motion signals” e.g., accelerometer signals) may include components that are due to such subject motion, or “motion artifacts”. Motion artifacts may be potentially very large additive signal components in an accelerometer signal or in a gyroscope signal that interfere with the desired seismocardiogram or gyrocardiogram signals. These interfering signal components can negatively impact the ability to track instantaneous heart rate. As used herein, the terms “subject motion” and “motion artifacts” refer to motion of the inertial sensor (and to the corresponding signals produced by the inertial sensor) that is not due to cardiac motion or thermal noise. The term “cardiac motion” refers to motion (e.g., of an inertial sensor) caused by activity of the heart of the subject.

In some embodiments, various techniques are used to attenuate the interfering motion artifact signal components while preserving the desired signal components, thus enabling the tracking of instantaneous heart rate in the presence of subject motion. Such techniques may be used to improve the performance of external inertial sensors (worn by the subject) on the chest or neck of the subject or internal inertial sensors (implanted in the subject) in the chest or neck of the subject. An internal inertial sensor in the neck may be a neck-implanted MEMS system that may, for example, measure pulsing of blood through the carotid artery.

In seismocardiography, chest or neck-mounted or implanted accelerometers may measure mechanical vibrations due to cardiac events such as muscle contractions, blood-flow turbulence, or well as the opening and closing of heart valves. Analogously, in gyrocardiography, chest-mounted gyroscopes (e.g., three-axis gyroscopes) may record these same vibrations to produce instantaneous calculations of angular velocity along each axis. The resulting time-series (the seismocardiogram and gyrocardiogram) may be used to make inferences about cardiac state, particularly tracking instantaneous heart rate. While the seismocardiogram and gyrocardiogram are rich in cardiac information, they may also be highly vulnerable to motion artifacts induced by activities of daily living such as walking or even smaller-scale, unintended motions due to, for example, sitting in a chair.

The motion artifacts in accelerometer signals may exceed, in magnitude, the desired seismocardiogram signals by up to an order of magnitude or possibly more. Motion artifacts may seriously degrade the quality of seismocardiogram-based heart rate calculations or make direct calculation of heart rate from the motion-artifact corrupted signal impossible. Various approaches to motion-artifact cancellation may be used. These include linear filtering methods for performing frequency-selective filtering and wavelet denoising methods for obtaining both time and frequency selectivity. Other approaches based on the Empirical Mode Decomposition (EMD) may also be employed.

From an implementation perspective, one of the simplest approaches well-suited to real-time applications is that of a linear filter. This disclosure includes design strategies for the coefficients associated with such a filter and some related generalizations. Examples are presented in the context of accelerometer-based seismocardiogram signals, but the same or analogous concepts may be applied to gyroscope-based gyrocardiogram signals.

In some embodiments, a three-axis accelerometer is oriented such that the desired seismocardiogram signal is present only (or primarily) in one of the three axes (which may be the dorsal-ventral axis of the subject, and which may be referred to as the z-axis). Motion artifacts may be present in all three axes. Subject motion as simple as very gentle walking may generate signal components significantly larger in amplitude than the desired z-axis seismocardiogram signal which contains cardiac state information. In some embodiments, the three-axis accelerometer is oriented such that one of the accelerometers is oriented parallel to the dorsal-ventral axis of the subject. As used herein, a sensor including a plurality of sensors (e.g., a three-axis accelerometer including three accelerometers or a three-axis magnetometer including three magnetometers) may be referred to as a “compound” sensor, and a sensor consisting of a single sensor (e.g., a single-axis accelerometer) may be referred to as a “simple” sensor.

For an inertial sensor worn by the subject on the chest of the subject, the desired seismocardiogram signal may be present only (or primarily) in the signal parallel to the dorsal-ventral axis. In some embodiments, the (three-axis) accelerometer is instead oriented so that none of the axes of the accelerometers is parallel to the dorsal-ventral axis of the subject; in such an embodiment, the signals from the simple accelerometers may be multiplied by a suitable rotation matrix so that one of the signals in the resulting product vector is sensitive only to acceleration parallel to the dorsal-ventral axis of the subject.

In some embodiments, finite impulse response (FIR) filtering is used to combine the three signals ax (t), ay (t), and az (t) from a three-axis accelerometer. For example each of the signals may be filtered with a respective FIR filter, and the outputs of the three filters may be summed, so that during each time increment the summed output is a weighted sum of (i) the samples of ax (t) in a first time window (ii) the samples of ay (t) in a second time window and (iii) the samples of az (t) in a third time window. The weights of this sum may be referred to as “filter coefficients”. Each of the time windows may (i) start before the time t for which the seismocardiogram is being calculated, or (ii) end after the time t. If one or more of the time windows ends after time t then this creates processing delay. The window start and end times may in general be different for each axis.

Various design strategies may be employed to select the filter coefficients. For example, because motion artifacts in each of the three axes may exhibit some degree of mutual correlation, the filter coefficients may be selected to perform linear prediction (and subsequent, partial cancellation) of z-axis subject motion from x and y axes motion signals. As such, filter coefficients may be designed such that the sum of the filter outputs approximates the z-axis output; because the cardiac motion component may be substantially absent from the x-axis and y-axis signals, such filter coefficients may also cause the x-axis and y-axis filter outputs to approximate the motion artifact component of the z-axis output. Various optimization criteria may be used to find the coefficients. For example, the coefficients may be chosen to minimize the sum of the square of the differences between z-axis output and the summed x and y axes filter outputs. Once the coefficients have been determined (using this method or another method) they may be fixed and they may continue to be used in operation, per patient, or for a plurality of patients. Alternatively, data may be collected over some finite interval, and the filter coefficients may be calculated based on the windowed data. These coefficients may then be held fixed for some finite duration before being updated again. Gradient descent may be used for such an optimization (to determine the filter coefficients), or the coefficients may be determined by solving a set of linear equations associated with a least squares optimization criterion. For example, data may be collected from the three-axis accelerometer during an interval of time (e.g., during a 20-second interval, or during an interval having a length between 5 seconds and 100 seconds) and filter coefficients may be found using a suitable optimization method (e.g., a gradient descent method) by adjusting the coefficients, in the filters used for the signals ax (t) and ay (t), in a direction that reduces the mean squared signal in the sum of the three filtered signals. In some embodiments, the adjusting of the filter coefficients is implemented recursively via, e.g., a Least Mean Squares (LMS) algorithm (discussed in further detail below).

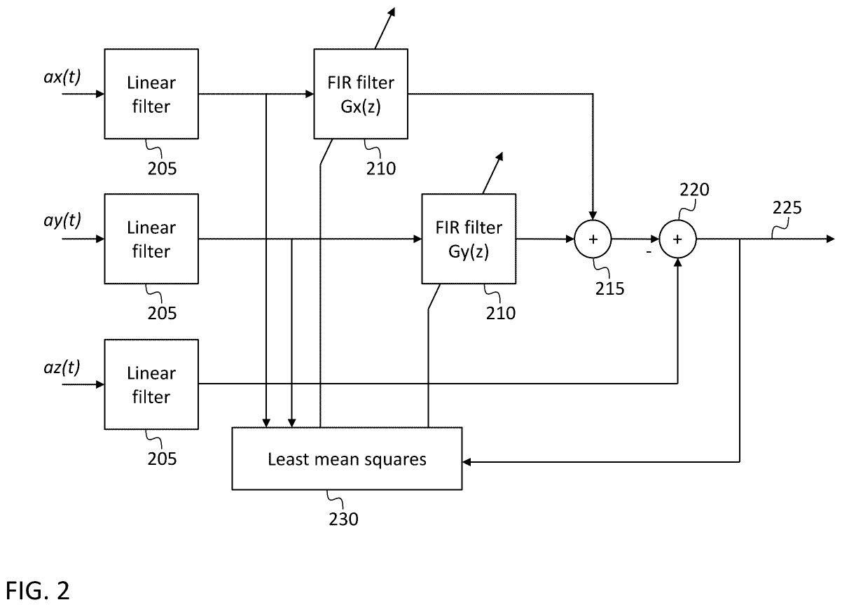

FIG. 2 is a block diagram of a set of filters that may be used to perform suppression of subject motion components in the z-axis acceleration signal. In this embodiment, the predicted z-axis motion is subtracted from the z-axis signal to achieve partial subject motion cancellation in the z-axis. All three acceleration signals ax (t), ay (t), and az (t) may undergo optional linear filtering, using linear filters 205 with transfer function H (z). The desired seismocardiogram signal and the motion artifacts may overlap in the frequency domain, but it is possible that their spectra differ; for example the motion artifacts may have a spectrum extending to DC whereas the seismocardiogram signal may have a spectrum extending from a lower frequency (which may correspond to a minimum heart rate) to an upper frequency. As such, H (z) may be a simple high-pass filter with cut-off frequency chosen to reject as much of the motion artifact as possible while preserving as much as possible of the seismocardiogram signal.

In some embodiments, some or all of the residual z-axis motion artifact signal is removed from the z-axis signal by exploiting the correlation of the z-axis motion artifact signal with the x and y axis motion artifact signals. For example, a first FIR filter 210, with transfer function Gx (z), may be used to filter the x-axis signal, or a second FIR filter 210, with transfer function Gy (z) may be used to filter the y-axis signal, or both may be used to filter the x and y axis motion artifact signals, such that the sum of the filter outputs (which my be generated by a first adder 215) approximates (mutually correlated components in) the z-axis subject motion component. The output of the first adder 215 may then be subtracted (by a second adder 220) from the z-axis signal to form an output signal 225 in which the effect of motion artifacts may be suppressed and which may therefore be better suited as a seismocardiogram signal (e.g., for the calculation of the instantaneous heart rate) than the raw z-axis acceleration signal.

An adaptive algorithm may be used to update the filter coefficients in response to changes in subject motion signal characteristics. This updating is represented by the diagonal arrows in FIG. 2. In FIG. 2, and in other drawings of this disclosure, a diagonal arrow extending through a block represents parameter-control information, e.g., a signal, or information, used to adjust the behavior of the block. For example, if the block includes a FIR filter, then the diagonal arrow may represent filter weights supplied to the filter, or one or more signals based on which the filter weights may be adjusted. One candidate update procedure is the least mean squares (LMS) algorithm. In this context, the subject-motion-cancelled seismocardiogram calculation acts as the error signal driving the LMS update of the filter coefficients. LMS adjusts coefficients to minimize a measure of the magnitude of the output signal 225 (e.g., to minimize the power of the output signal 225). The LMS method may include, starting from an initial set of parameter values (e.g., weights of the FIR filters 210), (i) receiving input data (e.g., from the linear filters 205), (ii) generating an output signal (e.g., the output signal 225) (iii) calculating an error (e.g., a measure of the output signal 225), and (iv) updating the parameter values (e.g., the weights of the FIR filters 210), based on the error and the input signal.

In some embodiments, as illustrated in FIG. 2, a shared LMS element 230 (e.g., an LMS circuit, or an LMS algorithm) receives the signals from x and y axes, and the output signal 225, and adjusts the parameters of the two FIR filters 210 so as to minimize (e.g., the power of) the output signal 225.

In some embodiments, whitened matched filtering may be employed to generate a seismocardiogram signal, from which the times of quasi-periodic cardiac events (e.g., the aortic valve opening time (AVOT)) may be calculated. The successive differences of these times may serve as calculations of the beat interval. If the shape of the transient pulse, in the seismocardiogram signal, around the aortic valve opening time is known a-priori (such a pulse shape may be referred to as a “model cardiac motion signal”), a length N FIR filter with coefficients matched directly to the pulse can be applied such that the times of the highest maxima of the filter output correspond closely to the aortic valve opening times. Under a white Gaussian noise assumption, these aortic valve opening time calculations may be optimum in a mean-squared-error sense. Thus, successive differences of the calculated aortic valve opening times may serve as high quality calculations of the beat intervals. Also, under the same white Gaussian noise assumption, such filters may maximize SNR when temporally aligned with the transient pulse.

In the presence of subject-motion-induced noise, the noise assumptions may not be valid, in part because motion artifacts may be non-stationary and non-Gaussian. However, it may still be possible to generalize the matched filter by pre-whitening the received signal based on (at least local) measures of the N×N noise covariance matrix, R. The pre-whitening may be implemented, for example, by left-multiplying the original vector of FIR coefficients corresponding to the aortic valve opening pulse samples by the inverse of the sample covariance matrix of the motion artifacts. If the sample covariance matrix of the motion artifacts is not readily available, then the full data covariance matrix may be used to approximate the motion artifact data covariance, if the desired signal component is much smaller than the motion artifact. The covariance matrix may be updated in real time. As such, changes in the covariance matrix may result in changes to the filter, causing the resulting filter to be adaptive.

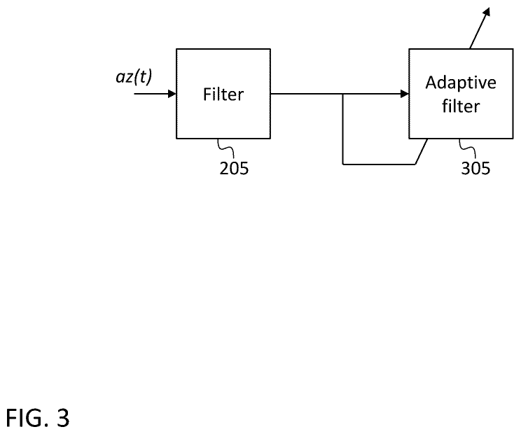

A block diagram of a whitened matched filter is shown in FIG. 3. The raw z-axis measurements az (t) are optionally processed by a fixed filter 205 (designed to remove mostly out-of-signal-band noise (as described in the previous section). The adaptive filter 305 may accept parameter-control information in the form of a seismocardiogram template sequence around the aortic valve opening time. When the subject is stationary, such a sequence can be computed directly as an average of segments of accelerometer data centered around aortic valve opening peaks. The adaptive filter 305 may further accept parameter-control information to perform whitening of the motion artifact component of the signal.

Whitening may use a full rank covariance matrix. However, the presence of the fixed filter 205 as well as the general low pass nature of the motion artifacts may cause the covariance matrix to be poorly conditioned, which may cause inversion of the matrix to be numerically unstable. This may be remedied by adding a regularization term to the covariance R; the regularization term may be the product of a small positive constant and an N-dimensional identity matrix (e.g., the regularization term may be a scaled identity matrix having the same dimensions as R). In other embodiments the pseudo-inverse of (a low rank approximation to) R is used instead of the inverse of R.

In some embodiments, multi-scenario, offline learning of filter coefficients is used, with desired signal aiding. In some embodiments, a desired seismocardiogram signal is obtained from a subject at rest, and a motion artifact signal, with no (or a negligibly small) cardiac-related signal component is also obtained. Sums of these signals are generated to create an ensemble of simulated subject-motion-corrupted received signals. Subject motion cancellation filter coefficients are then derived to give good performance over the ensemble of synthesized signals. In other embodiments, the simulated subject-motion-corrupted received signals are categorized by how significant the subject motion is and filter coefficients for each category are derived. The different categories may have different peak amplitudes or standard deviations. In operation, tests of peak amplitude or standard deviation (or both) may be implemented to choose the appropriate filter. In the configuration of FIG. 3, the adaptive filter 305 may then be selected based on the detected peak amplitude or standard deviation of the accelerometer signal.

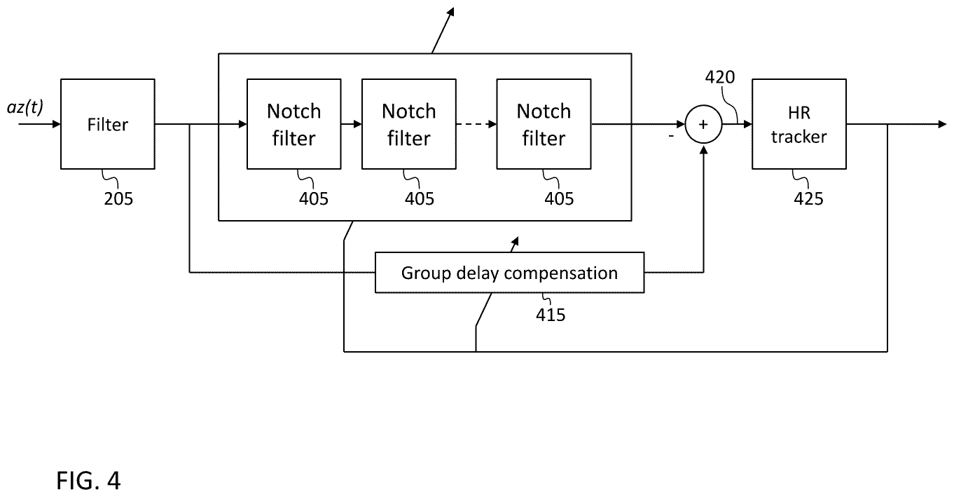

In some embodiments, a harmonic notch filter or fundamental frequency tracker is employed to suppress the component of the signal due to subject motion. Over a few heartbeats, the seismocardiogram may be quasi-periodic, and its energy may be confined to multiples of the fundamental frequency (which equals 1/60 of the heart rate (in beats per minute), over those few seconds). This fundamental frequency may be referred to as the “heart rate frequency”. In this case, a calculation of the motion artifact can be obtained as the output of a cascade of L notch filters, tuned to L respective harmonics of the fundamental frequency (with L being a positive integer), the cascade of notch filters being configured to receive, as input, a measured motion signal. A block diagram of a system for performing this filtering is shown in FIG. 4. As the term “harmonic” is used herein, the fundamental frequency (e.g., the heart rate frequency) is also a harmonic (e.g., the first harmonic) of the heart rate frequency.

In the embodiment of FIG. 4, a fixed filter 205 removes out-of-band motion artifacts. The resulting signal is then passed, for example, through a sequence of second-order infinite impulse response (IIR) filters 405 each tuned to notch out (e.g., suppress) a respective harmonic of the desired seismocardiogram, based on a previous calculation of the heart rate. In some embodiments, FIR filters are used. An IIR filter 405 may be capable of providing a similar notch width (to that of a candidate FIR filter) at lower computational complexity. The width of the notches may be adjusted to take into consideration the amount of uncertainty in the heart rate calculation. A calculation 420 of the desired signal is formed by subtracting the calculated motion artifact obtained at the output of the sequence of notch filters from an appropriately group-delay compensated version of the signal at the input to the notch filters. Group delay compensation may be performed by a linear group delay filter 415. If linear phase FIR notch filters are used, the group delay filter 415 may be a simple delay. In general, the group delay filter 415 may implement full (all-pass) group delay equalization for the nonlinear phase characteristics that may be associated with IIR notch filters. The group delay compensation may be a function of the notch filter frequency responses. Because the notch filters may change with time, the group delay compensation may also change with time, e.g., the characteristics or parameters of the group delay filter 415 may be adjusted, during operation, based on the output of the heart rate tracker 425 (discussed in further detail below), as shown, in FIG. 4, by the diagonal arrow extending through the block representing the group delay filter 415. A heart rate tracker 425 may receive the difference 420 between (i) the input from the inertial sensor and (ii) the calculation (produced by the sequence of notch filters 405) of the subject motion signal. The heart rate tracker 425 may then use this signal (which may be a seismocardiogram that is relatively free of motion artifacts) to generate a heart rate calculation.

In some embodiments, design criteria which promote reduced complexity filtering are employed. For example, the optimization criteria used in deriving the FIR filter coefficients may be modified so as to promote zero-valued coefficients. For example, Lasso regression, which includes a penalty term proportional to the sum of the absolute value of the filter coefficients, may be used. This penalty term may promote zero-valued coefficients, which reduce the computational complexity of the filtering operation.

Embodiments of the present disclosure may generate heart rate calculations, which may be used for various purposes. For example, the calculated heart rate may be reported (e.g., via a display, or via a wireless signal received by another device (e.g., a mobile device such as a mobile telephone or a portable (e.g., laptop) computer), which may display or log (e.g., in the cloud) the heart rate data). In some embodiments, changes in the heart rate (e.g., tachycardia) may be reported to a user (e.g., a clinician, or the subject) or used to initiate an intervention (e.g., a change in medication (e.g., in a patient connected to an intravenous delivery system) or electrical stimulation of the vagus nerve (if the change in heart rate is an indication that the subject may be experiencing an epileptic seizure)).

Embodiments of the present disclosure may generate heart rate calculations that are more accurate or reliable than heart rate calculations generated (e.g., based on inertial sensor signals) by other systems; as such, some such embodiments of the present disclosure improve the technology of removing motion artifacts from inertial sensor signals and improve the technology of heart rate calculation based on inertial sensor signals.

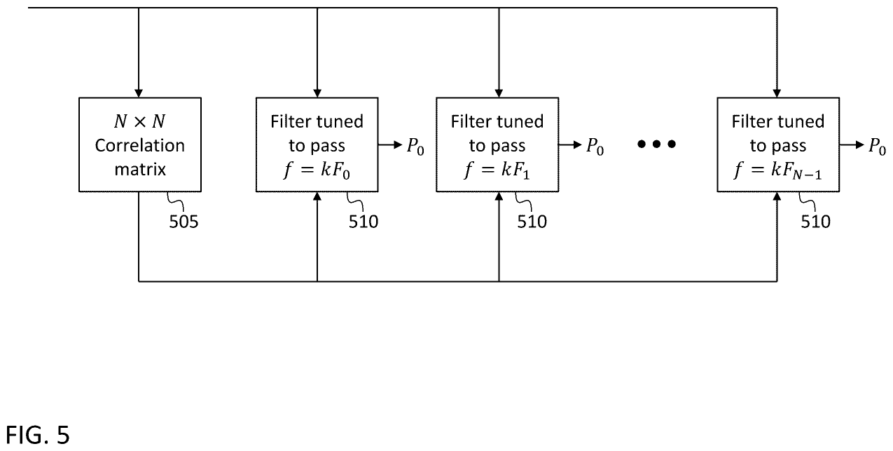

Processing methods described herein may be combined (e.g., cascaded) in various ways. For example, the filtering approach of FIG. 3, or the notch filter approach of FIG. 4, or the configuration of FIG. 5, may (i) be applied to one or more measured motion signals, or (ii) any of these approaches may be cascaded with the configuration of FIG. 1, e.g., any of these approaches may serve as a refinement after the combining of the signals over the three axes. Although some examples discussed herein discuss the use of an accelerometer to generate a seismocardiogram, the present disclosure is not limited to such embodiments. For example, a gyroscope may be used in an analogous manner (e.g., with analogous systems and methods for reducing the effects of motion artifacts) to generate a gyrocardiogram, or a combination of a gyroscope and an accelerometer may be used to generate (e.g., with analogous systems and methods for reducing the effects of motion artifacts) a signal that responds to cardiac motion, and that may be used, for example, to calculate the heart rate.

The notch filtering approach for motion cancellation relies on some knowledge of heart rate, which is a fundamental quantity of interest. This suggests that joint processing may be performed to obtain the heart rate and to suppress motion in the seismocardiogram. Such a joint processing approach may be used when the magnitude of the motion artifacts is sufficiently small that the algorithm does not incorrectly identify the frequency of periodic components of the motion as the heart rate.

In some embodiments, heart rate is calculated while suppressing relatively small motion using a bank of filters each tuned to a different candidate fundamental frequency (e.g., each tuned to a candidate heart rate). Each filter in such a bank of filters may be designed to have a transfer function with an amplitude of one at the center frequency of the passband (and at each harmonic of this center frequency), and to produce the minimum output power when used to filter the input signal. The effect of such a constrained minimization may be to suppress out of band signals (e.g., motion signals that are at frequencies different from the candidate heart rate).

In some embodiments, a bank of filters is used in which each filter is tuned to a different hypothesized fundamental frequency and each filter is adjusted, during operation, to suppress the signal (e.g., noise and motion components) at all frequencies other than at the hypothesized fundamental frequency of the filter and a fixed set of M−1 harmonics. This may be achieved through the computation of a calculated N×N signal covariance matrix. For single axis processing, such a matrix R (t) may be updated over time according to:

$$R ( t ) = α R ( t - 1 ) + ( 1 - α ) A ( t ) {A}^{T} ( t ) \qquad ( 1 )$$

$$A ( t ) = {[ a ( t ) , a ( t - 1 ) , … , a ( t - N + 1 ) ]}^{T} \qquad ( 2 )$$

where α is the input signal and a is a convergence rate parameter.

A minimum variance calculator may then be defined as a length N FIR filter

$$h = {[ {h}_{0} , {h}_{1} , … , {h}_{N - 1} ]}^{T}$$

that minimizes output power P=h^{T}Rh, subject to passing, with unit gain, tones at the fundamental frequency and its first M−1 harmonics. The design of the filter coefficients may be a function of the signal covariance R and may be a function of the assumed fundamental frequency. Each filter may be adaptive in the sense that the covariance matrix is a function of the second order statistical properties of the motion artifacts. Each filter may also be adaptive in the sense that the filter may be adjusted as the latest calculation of heart rate changes. For example, if the last good calculation of heart rate is 60 beats per minute (1 Hz) then each of the filters may be adjusted to pass a respective fundamental frequency at or near 1 Hz (and harmonics of this fundamental frequency). In other embodiments, a fixed grid of hypothesized fundamental frequencies is used instead. The filter lengths may be sufficiently short to avoid the risk that the adaptation process may result in unduly narrow passbands.

FIG. 5 shows such an embodiment, in which a circuit 505 for calculating a covariance matrix calculates the covariance matrix (e.g., according to equations (1) and (2) above) and feeds the result to each of a plurality of (e.g., N) filters 510 (e.g., filter circuits 510), each filter being tuned to pass a respective hypothesized fundamental frequency and a number of harmonics, and each filter being configured to adjust its coefficients based on the covariance matrix. The correlation matrix (shown in FIG. 5) may be equal to the covariance matrix. The calculated heart rate, in the embodiment of FIG. 5, may be the fundamental frequency of the filter, of the N filters, having the greatest output power. The circuits of FIG. 5 may be dedicated circuits, or general purpose processing circuits, or one or more of them may share a processing circuit, e.g., a general purpose processor running code to implement the functions shown in FIG. 5.

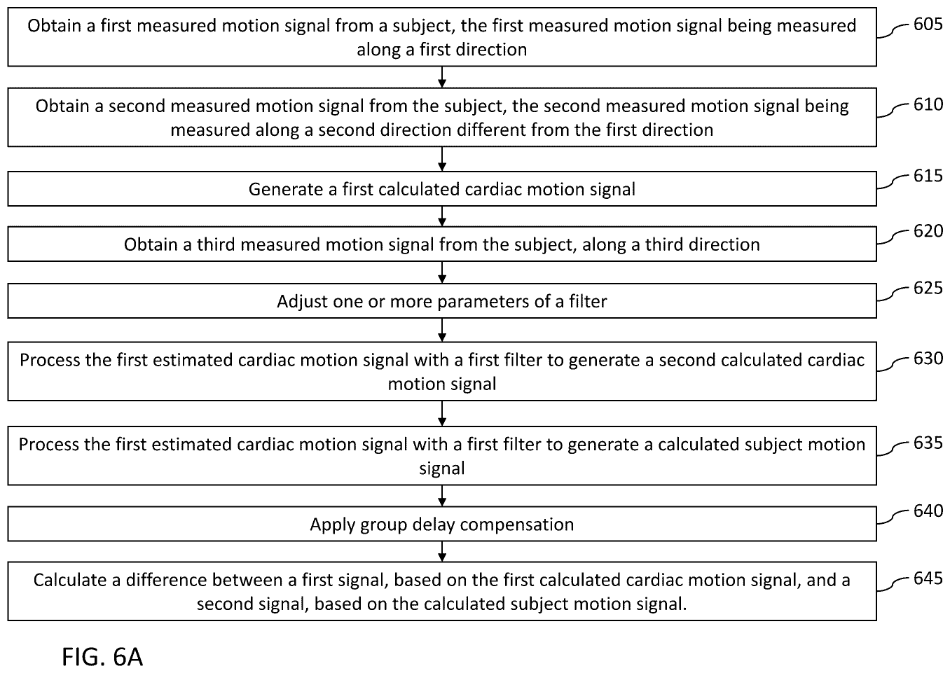

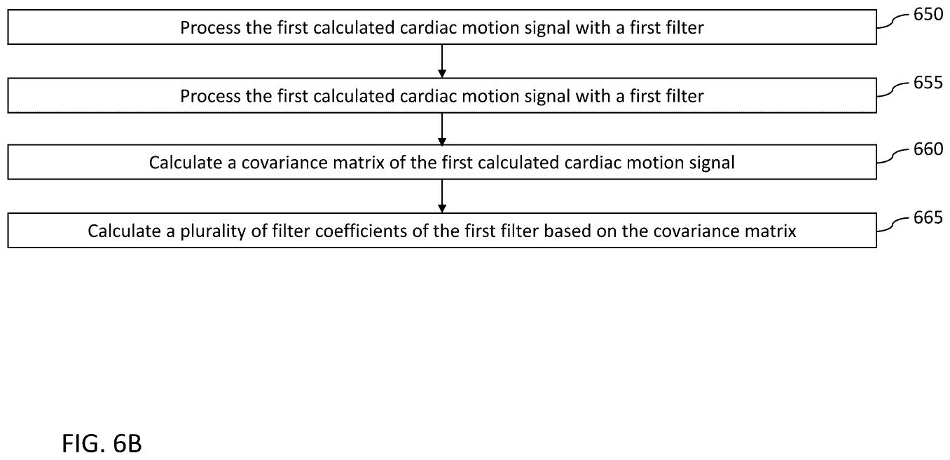

FIGS. 6A and 6B show a method, in some embodiments. Although FIGS. 6A and 6B illustrate various operations in a method of the present disclosure, embodiments according to the present disclosure are not limited thereto. For example, according to some embodiments, a method of the present disclosure may include additional operations or fewer operations, or the order of operations may vary (unless otherwise explicitly stated or implied) without departing from the spirit and scope of embodiments according to the present disclosure.

The method may include obtaining, at 605, a first measured motion signal from a subject, the first measured motion signal being measured along a first direction. For example, as discussed above, the first measured motion signal may be a signal from a first simple accelerometer of a three-axis accelerometer, the first simple accelerometer measuring acceleration along a first axis, the first axis being parallel to the dorsal-ventral axis of the subject. The method may further include obtaining, at 610, a second measured motion signal from the subject, the second measured motion signal being measured along a second direction different from the first direction. For example, as discussed above, the second direction may be perpendicular to the first direction, and the second measured motion signal may be a signal from a second simple accelerometer. As such, the second measured motion signal may include a significant component due to subject motion, but little or no contribution due to cardiac motion. The method may further include generating, at 615, a first calculated cardiac motion signal, the generating comprising combining the first measured motion signal and the second measured motion signal. For example, as discussed above in the context of FIG. 2, the component of the first measured motion signal due to subject motion may be suppressed by subtracting from the first measured motion signal a suitably processed (e.g., filtered) version of the second measured motion signal.

The method may further include obtaining, at 620, a third measured motion signal from the subject, along a third direction, the third measured motion signal being measured along a third direction different from the first direction and different from the second direction. For example, as discussed above, the first direction may be parallel to the dorsal-ventral axis of the subject, and the second and third directions may both be perpendicular to the z direction, and to each other. Like the second measured motion signal, the third measured motion signal may include a component correlated with the subject motion component of the first measured motion signal, and, as such, the third measured motion signal may, like the second measured motion signal, be suitably processed (e.g., filtered) and subtracted from the first measured motion signal to reduce the subject motion component of the first measured motion signal.

The method may further include adjusting, at 625, one or more parameters of a filter, e.g., of a filter used to filter the second measured motion signal before subtraction from the first measured motion signal, as discussed above in the context of FIG. 2. The adjusting may include adjusting the one or more parameters using least mean squares filtering of the first calculated cardiac motion signal. The method may further include processing, at 630, the first calculated cardiac motion signal with a first filter to generate a second calculated cardiac motion signal, where the first filter has characteristics related to a model cardiac motion signal and a calculated noise signal. For example, the first filter may include a whitening filter, for whitening the subject motion signal, and a matched filter, matched to the expected waveform of the cardiac motion signal. As used herein, a first filter may be said to “include” or “comprise” a second filter and a third filter if the first filter has the poles or zeros of the second filter and those of the third filter, regardless of whether the first filter is implemented as a cascade of the second filter and the third filter.

The method may further include processing, at 635, the first calculated cardiac motion signal with a first filter to generate a calculated subject motion signal. In such an embodiment, as discussed above in the context of FIG. 4, the first filter may include one or more notch filters, e.g., it may include a first notch filter for suppressing signal components at or near a first harmonic of a calculated heart rate frequency. The first filter may further include a second notch filter for suppressing signal components at or near a second harmonic of the calculated heart rate frequency. The method may further include applying, at 640, group delay compensation; and calculating, at 645, a difference between a first signal, based on the first calculated cardiac motion signal, and a second signal, based on the calculated subject motion signal. The method may further include processing, at 650, the first calculated cardiac motion signal with a first filter (e.g., a first one of the filters 510); and processing, at 655, the first calculated cardiac motion signal with a second filter (e.g., a second one of the filters 510). In some embodiments, the first filter is configured to pass a first harmonic of a first hypothetical heart rate, and the second filter is configured to pass a first harmonic of a second hypothetical heart rate. The method may further include calculating, at 660, a covariance matrix (e.g., by the circuit 505 for calculating the covariance) of the first calculated cardiac motion signal, and calculating, at 665, a plurality of filter coefficients of the first filter based on the covariance matrix (as discussed above in the context of FIG. 5).

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X-Y and the second quantity is at most X+Y. As used herein, when a second number is “within Y %” of a first number, it means that the second number is at least (1-Y/100) times the first number and the second number is at most (1+Y/100) times the first number. As used herein, the word “or” is inclusive, so that, for example, “A or B” means any one of (i) A, (ii) B, and (iii) A and B.

Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed herein could be termed a second element, component, region, layer or section, without departing from the spirit and scope of the inventive concept.

As used herein, the terms “use,” “using,” and “used” may be considered synonymous with the terms “utilize,” “utilizing,” and “utilized,” respectively.

It will be understood that when an element or layer is referred to as being “on”, “connected to”, “coupled to”, or “adjacent to” another element or layer, it may be directly on, connected to, coupled to, or adjacent to the other element or layer, or one or more intervening elements or layers may be present. In contrast, when an element or layer is referred to as being “directly on”, “directly connected to”, “directly coupled to”, or “immediately adjacent to” another element or layer, there are no intervening elements or layers present.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1-35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

It will be understood that when an element is referred to as being “directly connected” or “directly coupled” to another element, there are no intervening elements present. As used herein, “generally connected” means connected by an electrical path that may contain arbitrary intervening elements, including intervening elements the presence of which qualitatively changes the behavior of the circuit. As used herein, “connected” means (i) “directly connected” or (ii) connected with intervening elements, the intervening elements being ones (e.g., low-value resistors or inductors, or short sections of transmission line) that do not qualitatively affect the behavior of the circuit.

Although exemplary embodiments of a system and method for motion artifact mitigation in seismocardiography and gyrocardiography have been specifically described and illustrated herein, many modifications and variations will be apparent to those skilled in the art. Accordingly, it is to be understood that a system and method for motion artifact mitigation in seismocardiography and gyrocardiography constructed according to principles of this disclosure may be embodied other than as specifically described herein. The invention is also defined in the following claims, and equivalents thereof.

1. A method, comprising:
obtaining a first measured motion signal from a subject, the first measured motion signal being measured along a first direction;
obtaining a second measured motion signal from the subject, the second measured motion signal being measured along a second direction different from the first direction; and
generating a first calculated cardiac motion signal,
the generating comprising combining the first measured motion signal and the second measured motion signal.

2. The method of claim 1, wherein the first direction is substantially along a dorsal-ventral axis of the subject.

3. The method of claim 2, wherein the second direction is substantially perpendicular to the first direction.

4. The method of claim 3, further comprising obtaining a third measured motion signal from the subject, the third measured motion signal being measured along a third direction different from the first direction and different from the second direction,
wherein the generating comprises combining:
the first measured motion signal,
the second measured motion signal, and
the third measured motion signal.

5. The method of claim 1, wherein the combining of the first measured motion signal and the second measured motion signal comprises processing the second measured motion signal with a filter.

6. The method of claim 5, further comprising adjusting one or more parameters of the filter.

7. The method of claim 6, wherein the adjusting comprises adjusting the one or more parameters using least mean squares filtering based on the first calculated cardiac motion signal.

8. The method of claim 1, further comprising processing the first calculated cardiac motion signal with a first filter to generate a second calculated cardiac motion signal,
the first filter having characteristics related to:
a model cardiac motion signal; and
a calculated noise signal.

9. The method of claim 8, wherein the first filter comprises:
a whitening filter; and
a matched filter.

10. The method of claim 1, further comprising processing the first calculated cardiac motion signal with a first filter to generate a calculated subject motion signal, the first filter comprising a first notch filter for suppressing signal components at or near a first harmonic of a calculated heart rate frequency.

11. The method of claim 10, wherein the first filter comprises a second notch filter for suppressing signal components at or near a second harmonic of the calculated heart rate frequency.

12. The method of claim 10, further comprising:
applying group delay compensation; and
calculating a difference between a first signal, based on the first calculated cardiac motion signal, and a second signal, based on the calculated subject motion signal.

13. The method of claim 1, further comprising:
processing the first calculated cardiac motion signal with a first filter; and
processing the first calculated cardiac motion signal with a second filter, wherein:
the first filter is configured to pass a first harmonic of a first hypothetical heart rate; and
the second filter is configured to pass a first harmonic of a second hypothetical heart rate.

14. The method of claim 13, further comprising:
calculating a covariance matrix of the first calculated cardiac motion signal; and
calculating a plurality of filter coefficients of the first filter based on the covariance matrix.

15. The method of claim 14, wherein:
the calculating of the filter coefficients comprises calculating coefficients that minimize an output power, subject to passing, with unit gain, the first harmonic of the first hypothetical heart rate; and
the output power is a matrix product including, as factors, the covariance matrix, and a vector comprising the coefficients.

16. The method of claim 14, wherein the calculating of the covariance matrix comprises calculating the covariance matrix based on:
a previously calculated covariance matrix, and
samples of the first calculated cardiac motion signal.

17. A system, comprising:
an inertial sensor; and
a processing circuit,
the processing circuit being configured:
to obtain a first measured motion signal from the inertial sensor, the first measured motion signal being measured along a first direction;
to obtain a second measured motion signal from the inertial sensor, the second measured motion signal being measured along a second direction different from the first direction; and
to generate a first calculated cardiac motion signal,
the generating comprising combining the first measured motion signal and the second measured motion signal.

18. The system of claim 17, configured to be secured to a subject, with the first direction substantially along a dorsal-ventral axis of the subject.

19. The system of claim 18, wherein the second direction is substantially perpendicular to the first direction.

20. The system of claim 19, wherein the processing circuit is further configured to obtain a third measured motion signal from the inertial sensor, the third measured motion signal being measured along a third direction different from the first direction and different from the second direction,
wherein the generating comprises combining:
the first measured motion signal,
the second measured motion signal, and
the third measured motion signal.

21.-32. (canceled)
